(19) United States
(12) Patent Application Publication
(10) Pub. No.: US 2019/0142311 A1
(43) Pub. Date: May 16, 2019

(54) CALIBRATION FOR EAB BIOFLUID SENSORS

(71) Applicant: Eccrine Systems, Inc., Cincinnati, OH (US)

(72) Inventors: Jason C. Heikenfeld, Cincinnati, OH (US); Mikel Larson, Cincinnati, OH (US); Florika Macazo, Cincinnati, OH (US); Jacob A. Bertrand, Norwood, OH (US)

(73) Assignee: Eccrine Systems, Inc., Cincinnati, OH (US)

(21) Appl. No.: 16/163,011

(22) Filed: Oct. 17, 2018

Related U.S. Application Data
(63) Continuation-in-part of application No. 15/512,982, filed on Mar. 21, 2017, now abandoned.
(60) Provisional application No. 62/053,388, filed on Sep. 22, 2014.
(60) Provisional application No. 62/155,527, filed on May 1, 2015.

(51) Int. Cl. A61B 5/145; A61B 5/1495; A61B 5/00

(52) U.S. Cl. CPC A61B 5/14517; A61B 5/1495; A61B 2560/0223; A61B 5/6832; A61B 5/4266

(57) ABSTRACT

Devices for calibrating electrochemical aptamer-based biofluid sensors. In some embodiments a calibrant is located proximate to the EAB sensors, and calibration is facilitated by introduction of the biofluid into the device. Other embodiments include various mechanisms for introducing calibration solution to the sensors, including drawing calibration solution into contact with sensors through electrical potential, a dispensing mechanism such as an ink jet-type nozzle, as well as pressure actuated calibration dispensing. Also included are embodiments employing bifurcated biofluid transport paths that allow alternately for calibration and biofluid sensing.

### CROSS-REFERENCE TO RELATED APPLICATIONS

This application is a continuation-in-part of U.S. application Ser. No. 15/512,982, filed Mar. 21, 2017, and claims priority to PCT/US15/51439, filed Sep. 22, 2015, U.S. Provisional Application No. 62/155,527,filed May 1, 2015, and U.S. Provisional Application No. 62/053,388, filed Sep. 22, 2014, each of which is hereby incorporated by reference herein in its entirety.

### BACKGROUND OF THE INVENTION

Sweat sensing technologies have enormous potential for applications ranging from athletics, to neonates, to pharmacological monitoring, to personal digital health, to name a few applications. Sweat contains many of the same biomarkers, chemicals, or solutes that are carried in blood and can provide significant information enabling one to diagnose ailments, health status, toxins, performance, and other physiological attributes even in advance of any physical sign. Furthermore, sweat itself, the action of sweating, and other parameters, attributes, solutes, or features on, near, or beneath the skin can be measured to further reveal physiological information.

If sweat has such significant potential as a sensing paradigm, then why has it not emerged beyond decades-old usage in infant chloride assays for Cystic Fibrosis or in illicit drug monitoring patches? In decades of sweat sensing literature, the majority of medical literature utilizes the crude, slow, and inconvenient process of sweat stimulation, collection of a sample, transport of the sample to a lab, and then analysis of the sample by a bench-top machine and a trained expert. This process is so labor intensive, complicated, and costly that in most cases, one would just as well implement a blood draw since it is the gold standard for most forms of high performance biomarker sensing. Hence, sweat sensing has not emerged into its fullest opportunity and capability for biosensing, especially for continuous or repeated biosensing or monitoring. Furthermore, attempts at using sweat to sense “holy grails” such as glucose have not yet succeeded to produce viable commercial products, reducing the publicly perceived capability and opportunity space for sweat sensing.

Small, portable, and wearable biosensors are difficult to make such that they are precise and accurate. Such sensors are often generally challenged in their ability to make quality analytical measurements equal to what can be done with a dedicated measurement machine or large lab. This is especially true for sensors integrated in a small patch or wearable device because of the need for miniaturization and lower cost and because such devices are placed in less controllable environments than many lab or machine settings.

Many of the drawbacks stated above can be resolved by creating novel and advanced interplays of chemicals, materials, sensors, electronics, microfluidics, algorithms, computing, software, systems, and other features or designs, in a manner that affordably, effectively, conveniently, intelligently, or reliably brings sweat sensing technology into intimate proximity with sweat as it is generated. Further, a sweat sensor capable of analytical assurance is needed. With such a new invention, sweat sensing could become a compelling new paradigm as a biosensing platform.

Advanced sensor modalities, such as electrochemical aptamer-based biosensors (“EAB sensors”) require alternative calibration configurations and methods. Certain characteristics of EAB sensors, such as potential device-to-device variabilities at manufacture, sensor degradation or drift when exposed to biofluid, and differential response to variable biofluid potential of hydrogen (pH) or salinity, among others, recommend point of manufacture, point of use, and/or periodic calibration during use.

### SUMMARY OF THE INVENTION

The present invention provides a wearable sweat sensor device capable of analytical assurance. In one embodiment, a sweat sensor device with analytical assurance includes at least one sensor for detecting a first analyte, and at least one calibration medium containing at least the first analyte. When the first analyte in the at least one calibration medium comes into contact with the at least one sensor, the concentration medium provides a concentration calibration of the at least one sensor.

In another embodiment, a method of detecting a solute in sweat includes directing a calibration medium in a device to at least one sensor for detecting said solute in said device, calibrating said at least one sensor, positioning said device on skin, directing sweat to said device, and measuring said solute in said sweat using said device.

In another embodiment, a method of detecting a solute in sweat using a device for detecting said solute in sweat, said device including at least one sensor, includes providing fluidic access to said at least one sensor through an aperture in a first backing element by removing a second backing element from said device, directing at least one calibration medium to said at least one sensor through said aperture, calibrating said at least one sensor, placing said device on skin, directing sweat to said device, and measuring said solute in said sweat using said device.

The disclosed invention further provides devices and methods for calibrating electrochemical aptamer-based biofluid sensors. In some embodiments of the disclosed invention, a dry calibrant or calibration solution is located proximate to the EAB sensors, and calibration is facilitated by introduction of the biofluid into the device. Other embodiments include various mechanisms for introducing calibration solution to the sensors, including drawing calibration solution into contact with sensors through electrical potential, a dispensing mechanism such as an ink jet-type nozzle, as well as manually actuated calibration dispensing. Also included are embodiments employing bifurcated biofluid transport paths that allow alternately for calibration and biofluid sensing.

### BRIEF DESCRIPTION OF THE DRAWINGS

The objects and advantages of the present invention will be further appreciated in light of the following detailed descriptions and drawings in which:

FIG. 1A is a cross-sectional view of a device according to an embodiment of the present invention.

FIG. 1B is a cross-sectional view of the device of FIG. 1A during calibration.

FIG. 1C is a cross-sectional view of the device of FIG. 1 A positioned on skin.

FIG. 2A is a cross-sectional view of a device and a calibration module according to an embodiment of the present invention.

FIG. 2B is a cross-sectional view of the device and calibration module of FIG. 2A during calibration.

FIG. 2C is a cross-sectional view of a portion of the device of FIG. 2A.

FIG. 3 is a cross-sectional view of a device and a calibration module according to an embodiment of the present invention.

FIG. 4 is a cross-sectional view of a device according to an embodiment of the present invention.

FIG. 5 is a cross-sectional view of a device according to an embodiment of the present invention positioned on skin.

FIG. 6A is a cross-sectional view of a device according to an embodiment of the present invention positioned on skin.

FIG. 6B is a cross-sectional view of the device of FIG. 6A during calibration.

FIG. 6C is a cross-sectional view of a device according to an embodiment of the present invention positioned on skin.

FIG. 7A is a cross-sectional view of a device according to an embodiment of the present invention positioned on skin.

FIG. 7B is a cross-sectional view of the device of FIG. 7A during calibration.

FIG. 7C is a cross-sectional view of the device of FIG. 7A after calibration.

FIG. 8 is a cross-sectional view of a device according to an embodiment of the present invention.

FIGS. 9 and 9A depict at least a portion of an embodiment of the disclosed invention, wherein FIG. 9 is a view of a device without a biofluid flow, and FIG. 9A is a view of the device with a biofluid flow.

FIG. 10 depicts at least a portion of an embodiment of the disclosed invention.

FIG. 11 depicts at least a portion of an embodiment of the disclosed invention.

FIGS. 11A and 11B depict alternate embodiments of at least a portion of the disclosed invention as shown in FIG. 11.

FIG. 12 depicts at least a portion of an embodiment of the disclosed invention.

FIGS. 12A, 12B, 12C, and 12D depict alternate embodiments of at least a portion of the disclosed invention as shown in FIG. 12.

FIG. 13 depicts at least a portion of an embodiment of the disclosed invention.

FIG. 13A depicts an alternate embodiment of at least a portion of the disclosed invention as shown in FIG. 13.

### DEFINITIONS

As used herein, “sweat” means a biofluid that is primarily sweat, such as eccrine or apocrine sweat, and may also include mixtures of biofluids such as sweat and blood, or sweat and interstitial fluid, so long as advective transport of the biofluid mixtures (e.g., flow) is primarily driven by sweat.

As used herein, “biofluid” may mean any human biofluid, including, without limitation, sweat, interstitial fluid, blood, plasma, serum, tears, and saliva.

“Biofluid sensor” means any type of sensor that measures a state, presence, flow rate, solute concentration, solute presence, in absolute, relative, trending, or other ways in a biofluid. Biofluid sensors can include, for example, potentiometric, amp erometric, impedance, optical, mechanical, antibody, peptide, aptamer, or other means known by those skilled in the art of sensing or biosensing.

“Analyte” means a substance, molecule, ion, or other material that is measured by a biofluid sensing device.

“Measured” can imply an exact or precise quantitative measurement and can include broader meanings such as, for example, measuring a relative amount of change of something. Measured can also imply a binary or qualitative measurement, such as ‘yes’ or ‘no’ type measurements.

“Chronological assurance” means the sampling rate or sampling interval that assures measurement(s) of analytes in biofluid in terms of the rate at which measurements can be made of new biofluid analytes emerging from the body. Chronological assurance may also include a determination of the effect of sensor function, potential contamination with previously generated analytes, other fluids, or other measurement contamination sources for the measurement(s). Chronological assurance may have an offset for time delays in the body (e.g., a well-known 5- to 30-minute lag time between analytes in blood emerging in interstitial fluid), but the resulting sampling interval is independent of lag time, and furthermore, this lag time is inside the body, and therefore, for chronological assurance as defined above and interpreted herein, this lag time does not apply.

“EAB sensor” means an electrochemical aptamer-based biosensor that is configured with multiple aptamer sensing elements that, in the presence of a target analyte in a biofluid sample, produce a signal indicating analyte capture, and which signal can be added to the signals of other such sensing elements, so that a signal threshold may be reached that indicates the presence of the target analyte.

“Multi-capture Aptamer Sensor” means an EAB sensor capable of a plurality of analyte capture interactions, as disclosed in U.S. Pat. Nos. 7,803,542 and 8,003,374, which are hereby incorporated by reference herein in their entirety.

“Docked aptamer EAB sensor” means an EAB sensor that employs docking strategies to connect analyte capture complexes with the sensor electrode, and wherein such analyte capture complexes are configured for one analyte capture interaction, as disclosed in PCT/US18/39274, which is hereby incorporated by reference herein in its entirety.

“Sensitivity” means the change in output of the sensor per unit change in the parameter being measured. The change may be constant over the range of the sensor (linear), or it may vary (nonlinear).

“Signal threshold” means the combined strength of signal-on indications produced by a plurality of aptamer sensing elements that indicates the presence of a target analyte.

“Time-to-threshold” means the amount of time required for an EAB sensor to reach signal threshold. Such time may be calculated from the initiation of device use, the initiation of sweating, a sensor regeneration time, or other suitable starting point.

### DETAILED DESCRIPTION OF THE INVENTION

The present application has specification that builds upon International Application Nos. PCT/US13/35092, filed Apr. 2, 2013, PCT/US14/61083, filed Oct. 17, 2014, PCT/US14/61098, filed Oct. 17, 2014, PCT/US15/32830, filed May 28, 2015, PCT/US15/32843, filed May 28, 2015, PCT/US15/32866, filed May 28, 2015, PCT/US15/32893, filed May 28, 2015, and PCT/US15/40113, filed Jul. 13, 2015, the disclosures of which are hereby incorporated herein by reference in its entirety.

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

Embodiments of the present invention apply at least to any type of sweat sensor device that measures sweat, sweat generation rate, sweat chronological assurance, its solutes, solutes that transfer into sweat from skin, a property of or things on the surface of skin, or properties or things beneath the skin. Embodiments of the present invention further apply to sweat sensing devices that have differing forms including patches, bands, straps, portions of clothing, wearables, or any suitable mechanism that reliably brings sweat stimulating, sweat collecting, and/or sweat sensing technology into intimate proximity with sweat as it is generated. While certain embodiments of the present invention utilize adhesives to hold the device near the skin, other embodiments include devices held by other mechanisms that hold the device secure against the skin, such as a strap or embedding in a helmet.

Sweat stimulation, or sweat activation, can be achieved by known methods. For example, sweat stimulation can be achieved by simple thermal stimulation, by orally administering a drug, by intradermal injection of drugs such as methylcholine or pilocarpine, and by dermal introduction of such drugs using iontophoresis. Sweat can also be controlled or created by asking the subject using the patch to enact or increase activities or conditions which cause them to sweat. These techniques may be referred to as active control of sweat generation rate.

Certain embodiments of the present invention show sensors as simple individual elements. It is understood that many sensors require two or more electrodes, reference electrodes, or additional supporting technology or features which are not captured in the description herein. Sensors are preferably electrical in nature, but may also include optical, chemical, mechanical, or other known biosensing mechanisms. Any suitable sensor may be used in the disclosed invention (e.g., ion-selective, enzymatic, antibody, aptamer, optical, electrical, mechanical, etc.). Sensors can be in duplicate, triplicate, or more, to provide improved data and readings. Sensors may be referred to by what the sensor is sensing, for example: a sweat sensor; an impedance sensor; a sweat volume sensor; a sweat generation rate sensor; and a solute generation rate sensor.

The disclosure applies to sweat sensing devices with various configurations including patches, bands, straps, portions of clothing, wearables, or any suitable mechanism that reliably brings sweat stimulating, sweat collecting, and/or sweat sensing technology into intimate proximity with sweat as it is generated. Some embodiments use adhesives to hold the device near the skin, but devices may also be secured by another suitable mechanism, such as a strap or helmet suspension. Certain embodiments of the disclosed invention show sub-components that may require additional obvious sub-components for use of the device in various applications (such as a battery), and for purpose of brevity and focus on inventive aspects are not explicitly shown in the diagrams or described in the embodiments of the present disclosure. As a further example, many embodiments of the disclosed invention may benefit from mechanical or other means to keep the devices or sub-components firmly affixed to skin or to provide pressure facilitating constant contact with skin or conformal contact with ridges or grooves in skin, as are known to those skilled in the art of wearable devices, patches, bandages, or other technologies or materials that are affixed to skin. Such means are included within the spirit of the disclosed invention.

In an aspect of the present invention, a sweat sensor device is capable of providing analytical assurance as described below. Analytical assurance means (but is not limited to) an assurance of the precision, accuracy, or quality of measurements provided by the sweat sensor device. In other words, analytical assurance could further refer to an improvement in the confidence of the precision, accuracy, or quality of measurements made.

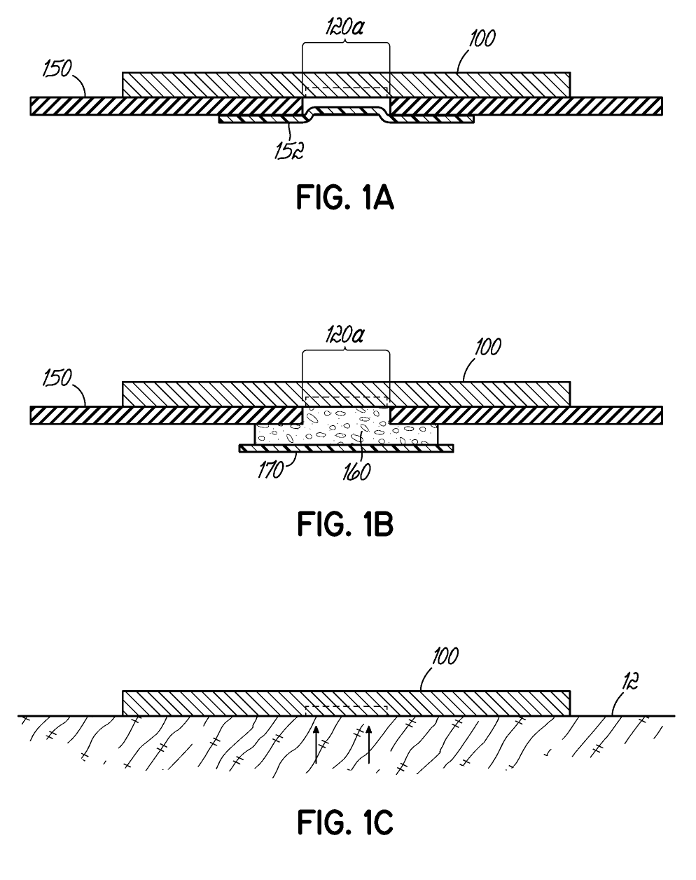

With reference to FIGS. 1A-1C, a sweat sensor device is designed to be calibrated before use. The sweat sensor device 100 has an adhesive side supported by carrier 150 and carrier 152. Carriers 150, 152 could be a variety of materials. By way of example, carriers 150, 152 could be wax or siliconized paper, such as that used in bandage backings. Carrier 150 is sufficiently sealed against the underside of the device 100 such that it covers and seals the adhesive side of the device 100 with exception to aperture 120a. Aperture 120a allows access to one or more sensors (not shown) of the device 100 to be accessed directly or through microfluidic connections. Carriers 150, 152 are removable from device 100. In the illustrative embodiment, the carrier 152 may be removed without removing the carrier 150.

With reference to FIG. 1B, the carrier 152 of the sweat sensor device 100 may be removed to expose the aperture 120a. A sponge 160, which is permeated with a calibrating solution or medium, is pressed against the device 100 to bring the solution in contact with the sensors of the device 100. Importantly, the carrier 150 shields the rest of the device 100 from the application of the calibrating solution but allows the calibration solution or medium to contact at least one sensor though the aperture 120a. The calibrating solution is provided with pre-determined concentration of solutes or other properties of sweat (e.g., pH). The sponge 160 is held against the device 100 for a period of time adequate such that the sensors can be calibrated based on readings of the solution. The amount of time required for a sensor to be calibrated may vary depending on the amount of time it takes for the sensor to stabilize. The length of time for a sensor to stabilize can be, for example, as short as several minutes, to as long as 30 minutes for nM or pM sensor, to as long as a number of hours for ion-selective electrodes that require wetting periods. Once the sweat sensor device 100 has completed calibration, it is now capable of providing analytical assurance. Carrier 150 may be subsequently removed, and the device 100 may be applied to skin 12 to be used, as shown in FIG. 1C. Advantageously, such a calibration technique improves the ease with which sensors in patches or wearable devices may be calibrated. Conventionally, sensors are calibrated by being dipped into a beaker or vial containing a solution of calibrating fluid. For a sensor in a patch or wearable device, as taught herein, such a technique is generally impractical for commercial usage (e.g. a non-laboratory setting such as a home) and is also not ergonomic.

A variety of techniques and compositions may be used to calibrate sensors according to methods of the present invention. For instance, a calibration solution the composition of which is based on properties of skin, contaminants on skin, or other solutes or properties that would affect analytical assurance for a sensor placed on skin may be used. A collected human sweat sample or an artificial sweat sample (e.g., such as one available from Pickering Laboratories) may also be used to calibrate a sensor. Further, the solution could be concentrated, diluted, or spiked with a solute or property of interest. The selected concentration of solutes could be, for example: low enough to confirm the lower limit of detection for the sensor or near; or below physiological levels to confirm the accuracy of the sensor. Where a device includes more than one sensor, the concentration of solutes in the applied sponge 160 could be designed to calibrate all of the sensors, one of the sensors, or a subset of the sensors. In an alternate embodiment, sponge 160 can be replaced by any other technique to apply a calibrating solution, including for example using a spray bottle (not shown).

In one embodiment, more than one calibration solution may be applied with similar or different concentrations or properties of sweat to calibrate a sensor. In the embodiment illustrated in FIG. 1B, more than one sponge 160 may be applied in sequence (not shown) to the device 100. Where more than one sponge 160 are applied in sequence, the sponges 160 may have concentrations of solutes, for example, that increase in concentration or properties to calibrate sensor response or linearity with change in concentration. Alternatively, the sponges 160 may have concentrations that increase or decrease to determine the rate of response or adaptation of sensors. Because some sensors have difficulty returning to their limit of detection as analytes do not quickly disassociate from the sensor as the concentration of the analyte decreases in solution, determining the rate of response improves the analytical assurance for the sensor. The application of a calibration solution (e.g., using the sponge 160) allows one to determine other properties such as drift of sensors over time. In one embodiment, a sponge 160 may be applied for a sufficient time such that sensor drift can be determined to improve the analytical assurance for the sensor. For high quality sensors, typically drift is observable only after a period of hours or more.

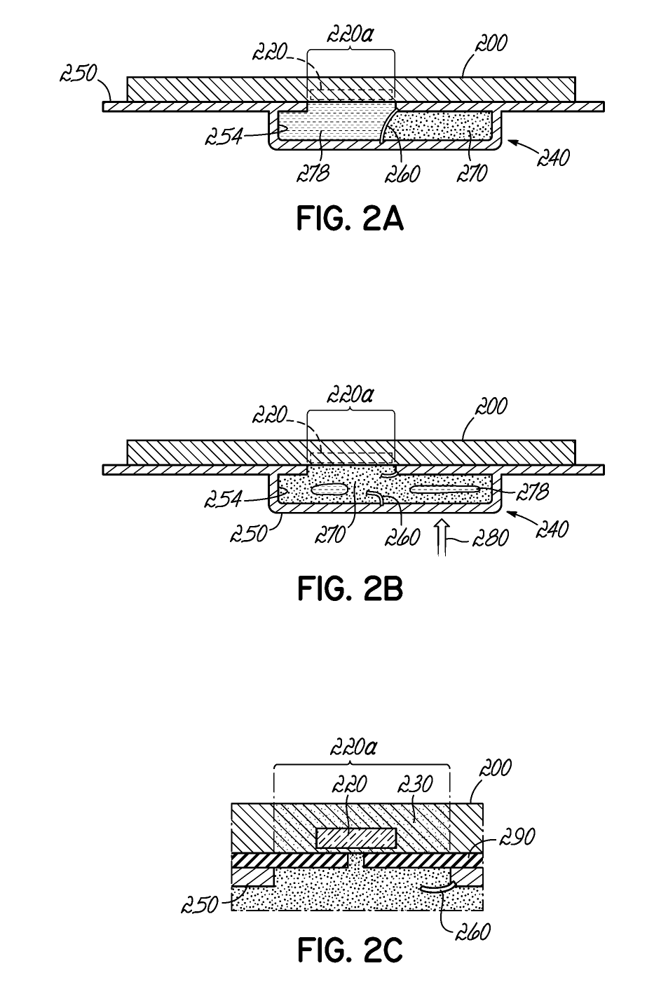

With reference to FIGS. 2A and 2B, a sweat sensor device 200 is coupled to a calibration module 240. The calibration module 240 includes a housing 250 that defines a reservoir 254. The calibration module 240 acts as a carrier for the device 200 similar to the carrier 150 of FIG. 1A. Housing 250 includes aperture 220a that provides fluidic access from the reservoir 254 to at least one sensor 220 (shown in FIG. 2C) within the device 200. A calibration solution 270 is sealed inside the housing 250 by a membrane 260. On the other side of the membrane 260 (i.e., the side of the reservoir 254 adjacent the aperture 220a) is a gas, inert gas, or fluid 278. The application of pressure (as indicated by arrow 280) to the housing 250 causes the membrane to rupture, as shown in FIG. 2B. In this regard, the calibration module 240 has been activated by the pressure applied in the direction of arrow 280 and the calibration solution 270 comes into contact with one or more sensors of the device 200 near aperture 220a. The pressure may be applied, for example, by a user pressing against the housing 250. In one embodiment, to ensure the sensors are wetted, the calibration module 240 may include a sponge material (not shown) on the side of the membrane 260 adjacent to the aperture 220a. Alternatively, the housing 250 may be designed such that gravity is not a factor in the movement of the calibration solution 270 past the sensor and/or that a shaking motion could be applied to ensure calibration 270 comes into contact with one or more sensors of the device 200.

In one embodiment, the device 200 may include a flow restricting element. As illustrated in FIG. 2C, the flow restricting element 290 may be positioned adjacent the aperture 220a between the device 200 and the housing 250. A wicking material 230 surrounds a sensor 220 and the restricting element 290. The flow restricting element 290 may be, for example, a flow limiting element, a flow constriction element, or a flow stopping element. In the illustrated embodiment, the restricting element 290 is a polymer film with a restriction in it, such as a small gap. In this configuration, the gap restricts the flow of sweat from the skin to wicking material 230. The flow restricting element may prevent a sweat pumping element, such as wicking material 230, within the device 200 from being saturated with the calibration solution 270. In other words, the sweat pumping capacity of device 200 is not saturated with the calibration solution 270. While the restricting element 290 is shown in FIG. 2C as being part of device 200, other configurations are possible. In one embodiment, the flow restricting element 290 could be a component of element 250 shown in FIGS. 2A and 2B.

With further reference to FIG. 2C, in one embodiment, element 230 may represent several layers of textile and the flow restricting element 290 may be a layer of paper with a low porosity and low flow rate. The layers of textile or paper may be, for example, 1 mm thick and 1″×3″ in area to allow wicking of sweat for a prolonged period of time. The paper layer 290, which is between the skin and the layers of textile 230, acts as the only fluidic connection point between skin and the layers of textile 230. In one embodiment, the paper layer 290 may have a relatively small area of 0.1″×0.1″. As a result, applying calibrating solution would not completely wet and saturate the pumping capacity of layers of textile or a wicking component. In one embodiment, pumping or wicking elements could be removed or not fluidically connected to sensors during calibration and added or connected after calibration is complete.

With further reference to FIGS. 2A and 2B, in one embodiment, the calibration solution 270 could be a gel and component 278 may be a gel (rather than the gas 278 discussed above). As membrane 260 ruptures, the calibration gel 270 comes in contact with the gel 278. The solutes in the calibration gel 270 will diffuse, rather than flow by advection, through the gel 278 to come into contact with one or more sensors of the device 200 near aperture 220a. The materials for gels 270, 278 could be similar gel materials or different, so long as the diffusion of solutes in gel 270 can occur through the gel 278. This configuration allows for calibration of the sensors over a varying concentration level. For example, a sensor could be calibrated between a zero concentration level which is the starting concentration for gel 270 and the maximum concentration of the solutes which results from slow diffusion-based mixing of concentrations between gel 270 and gel 278 where gel 278 contains a concentration of at least one solute to be used for calibration. Although such a calibration involving a concentration gradient could be achieved where components 270, 278 are liquids, such a calibration could be less predictable due to the mixing of the fluids being less predictable (e.g., is often more chaotic) than the diffusion of solutes where components 270, 278 are gels, which are more homogeneous.

With further reference to FIG. 2B, in one embodiment, the rupture of membrane 260 could be provided as the housing 250 is removed from the device 200. This may be more convenient as the device 200 cannot be adhered onto skin until housing 250 is removed. During the removal of the housing 250, the calibration solution 270 could be quickly (as little as seconds) brought into contact with sensors of the device 200, and the device may be applied to the skin. The calibration of the sensors may continue until sweat from the skin replaces the calibration solution, which is a process that may take at least several minutes, if not much longer. This approach ensures that the user always calibrates the device before use, without any extra steps beyond the expected minimum (i.e., removal of the housing 250) for applying an adhesive patch to the skin. This may be more broadly referred to as calibration which occurs as backing element or material, or housing material, is removed from the adhesive side of a device.

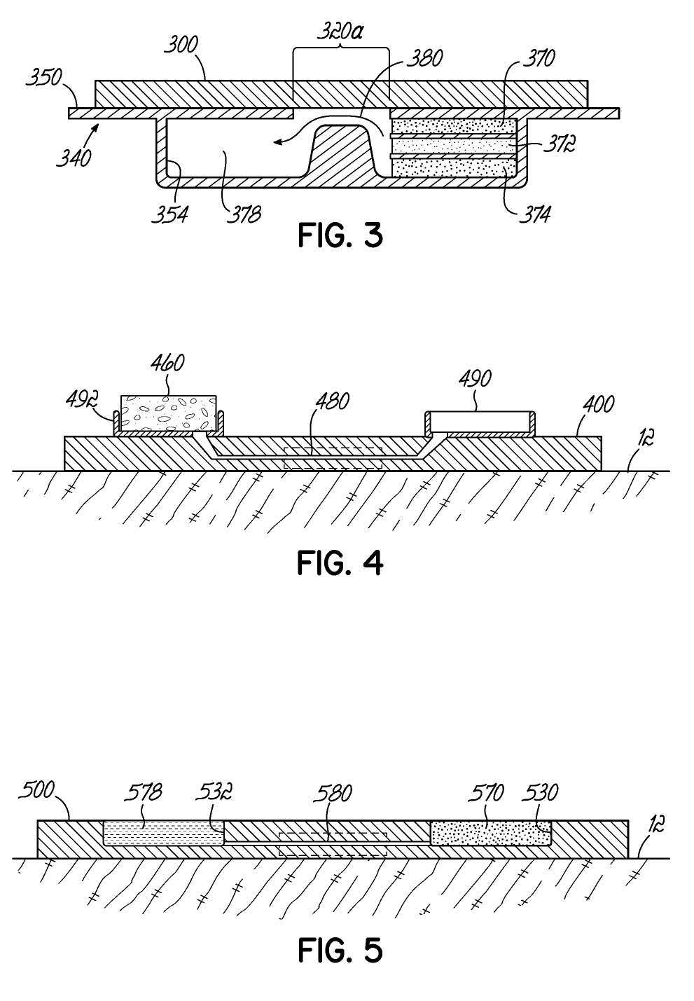

In one aspect of the invention, a calibration module may include more than one calibration solution or medium. With reference to FIG. 3, a device and a calibration module according to another embodiment of the invention are shown. The device 300 and calibration module 340 are similar in construction to that shown in FIGS. 2A and 2B, except as otherwise described below. In that regard, similar reference numerals refer to similar features shown and described in connection with FIGS. 2A and 2B. The calibration module 340 includes multiple solutions 370, 372, 374 within the reservoir 354. The solutions 370, 372, 374 could sequentially flow over aperture 320a past the sensors (as indicated by arrow 380) inside calibration module 340. The solutions 370, 372, 374 displace gas 378 as they flow past aperture 320a. The calibration module 340 may include a mechanism for pumping, gating, or introducing fluids as known by those skilled in the art. For example, component 378 could be a sponge material (not shown) that wicks the solutions 370, 372, 374 against the sensor. Further, the device 300 may include an electrowetting gate (not shown) to form a capillary between the solutions 370, 372, 374 and the sponge. It will be recognized that more complex arrangements with mechanical pumps and valves could be also used in embodiments of the present embodiment. The solutions 370, 372, 374 may have the same or varying concentrations. In one embodiment, the solutions 370, 372, 374 contain a lowest concentration, a middle concentration, and a highest concentration, respectively, for calibration.

In another aspect of the present invention, a calibration module may include one or more calibration solutions containing more than one solute. Such a configuration allows for the calibration of the sensors of a device, while also allowing for a determination of any cross-interference between various solutes in or properties of a sweat. For example, potassium (K) and ammonium (NH₄) are known to interfere with each other in ion-selective electrode sensors. In one embodiment, a calibration module (e.g., module 340) includes a first solution containing a high concentration of K and a low concentration of NH₄. A second solution in the calibration module may contain a low concentration of K and a high concentration of NH₄. Further solutions may contain equal concentrations of K and NH₄, which could be high, moderate, or low. In this manner, a determination may be made of any cross-interference between K and NH₄ for a device (e.g., device 300).

With reference to FIG. 4, device 400 includes an external introduction port 490, a microfluidic component 480 that moves fluid to or past sensors, and an optional outlet port 492 with absorbing sponge 460. Microfluidic component 480 may be, for example, a 50 micron polymer channel that is 500 microns wide. One or more calibration solutions could be introduced at port 490 while the device 400 is on the skin 12. The calibration solution may be introduced at port 490 using a variety of methods. For example, the calibration solution could be introduced at port 490 by the application of droplets, by using a cartridge, by using a carrier, such as those discussed above, or using another approach. In addition to a calibration solution, a fluid that refreshes the usability of sensors may also be introduced to the device 400 though port 490 and be wicked through the microfluidic component 480 across sensors by sponge 460. In various embodiments, the fluid may change the pH level or cause a sensor probe to release an analyte. In one embodiment, such a refreshing fluid could be introduced to the device 400, followed by the introduction of the calibration fluid. The introduction of a fluid (e.g., a calibration solution) may be followed by a removal of the fluid. For example, in one embodiment, the sponge 460 could be removed after collection of the refreshing fluid and disposed of. The sponge 460 could be a wicking sponge material, a textile, hydrogel, or other material capable of wicking and collecting a fluid.

With reference to FIG. 5, a device 500 a first reservoir 530 and a second reservoir 532 fluidically coupled by microfluidic component 580. The first reservoir 530 includes a calibrating solution 570, and the second reservoir 532 includes a displaceable gas 578. Microfluidics 580 is designed to provide access to a sensor (not shown). Calibration of the device 500 using aspects of the present invention could occur before device 500 operation begins, before sweat from skin 12 is sampled, or at times during the use of the device 500 using one more methods of timed microfluidic operation known by those skilled in the art. By way of example, the device 500 may include gates that swell (close) or dissolve (open) after prolonged exposure to a fluid. The gates (not shown) may be formed by a swellable polymer or a soluble salt or sugar, for example. The calibration solution 570 could stay in contact with the sensors for a determined period of time before it is removed. The calibration solution 570 may be removed, for example, by wicking or by pumping. Pumping may be accomplished through gas pressure (not shown) using the release of an internal pressurized gas source or generated gas source (e.g., electrolysis of water). Alternatively, the calibration solution 570 could reside in contact with sensors until it is replaced by sweat.

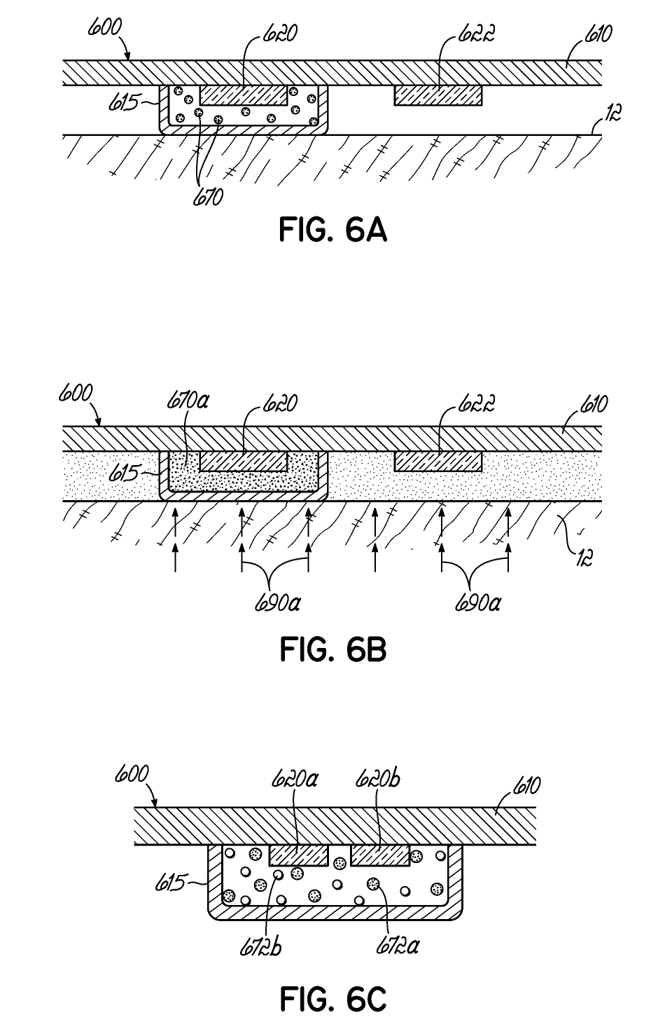

With reference to FIGS. 6A and 6B, a device 600 is shown which includes a substrate 610 carrying two similar sensors 620, 622 and a membrane 615 that covers the sensor 620. The sensors 620, 622 are similar in that, if one is calibrated, they are similar enough that calibration for one can be used for the others. In one embodiment, the sensors are of the same generation type (e.g. amperometric) but have different analyte targets (e.g. glucose and lactate). In another embodiment, the sensors target the same analyte, and calibration for one sensor will typically best predict the calibration for the second. Device 600 further includes a dry dissolvable calibration medium 670 for one or more analytes between the membrane 615 and the sensor 620. The calibration medium 670 could also be wet or a gel and need not necessarily be dry. FIG. 6B shows a flow of sweat 690 generated by the skin 12 as indicated by arrows 690a. The water in the sweat 690 penetrates through membrane 615 and dissolves calibration medium 670 to create a calibration solution 670a. Membrane 615 allows water transport through the membrane 615, while delaying or preventing transport of analytes to be sensed from the sweat 690 at least during a calibration between sensors. By way of example, the membrane 615 could be made of a dialysis membrane, Nafion membrane, track-etch membrane, reverse-osmosis membrane, or sealed reference electrodes. In this configuration, sensors 620, 622 can be compared in their readings of an analyte. If the concentration of an analyte in solution 670a is known, then the concentration of the analyte in sweat 690 can be better determined through comparison of the measured signal from sensors 620, 622. In an exemplary embodiment, membrane 615 creates a defined volume around sensor 620 such that the concentration of analytes is predictable (i.e., known amount of dilution as the calibration medium 670 dissolves). For example, a porous polymer or polymer textile could be used which has a finite porous volume in it to fix the volume of calibration solution 670a around the sensor 620. In one embodiment, calibration solution 670a may include a concentration of the analyte that is greater than the concentration of that analyte present in sweat. For example, the calibration solution 670a may include an analyte at a concentration roughly about 10 times or more than that found in the sweat that wets the calibrant.

With reference to FIG. 6C, in one embodiment, element 620 of the device 600 represents two or more different sensors 620a and 620b for calibration. For example, the first sensor 620a in element 620 could be for detecting cortisol, and often these types of sensors require calibration. Sensor 622 shown in FIG. 6A would, in this example, also be for detecting cortisol and would measure cortisol directly found in sweat. The second sensor 620b in element 620 could be for detecting Na (such as an ion-selective electrode or through simple electrical conductance of solution). The dry dissolvable calibration medium 670 includes a known starting concentration of cortisol 672a and Na 672b. As water moves through the membrane 615, it dilutes the calibration medium 670 to create the calibration solution 270a, in which concentrations of both Na and cortisol could be measured. The Na sensor 620b may be configured so that it would not need calibration using the calibration solution 270a. For example, sensor 620b may be an ion-selective electrode having a sealed reference electrode (not shown) to allow it to accurately quantify Na concentrations. As the Na dilutes as the water moves in, the amount of water is also indirectly measured (by measuring Na), and therefore the amount of dilution of cortisol would be known from the time when the water began moving through the membrane 615 until the water fills the space between the membrane 615 and the sensors 620a, 620b. In summary, the measurement of Na would be used to determine the total dilution that has occurred as water moves into the calibrating solution 670a, and therefore the amount of dilution of cortisol in calibrating solution 670a is also known. Therefore, a dilution calibration curve could be provided for the first sensor 620a, which would then provide a dilution calibration for sensor 622 as well.

With further reference to FIGS. 6A-6C, in one aspect of the present invention, membrane 615 may act as a binding medium that binds solutes in sweat such that sweat is diluted of one or more analytes before it reaches the calibrating medium. Such a binding medium would be in the sweat flow path between sweat glands and at least one sensor. The binding medium may provide specific binding (e.g., a layer of beads doped with ionophores) or non-specific binding (e.g., cellulose). As a result, the calibration medium 670 would not need to provide a concentration of analyte or analytes greater than that found in real sweat, as the initial sweat which reaches the calibration sensor would be diluted of the analyte to be calibrated. Specific binding materials include beads or other materials those known by those skilled in the art that promote specific absorption.

In another aspect of the present invention, conditions can be provided that denature or alter an analyte in sweat such that its concentration is effectively lowered before reaching a calibration medium. In one embodiment, a binding solute in solution that binds to the analyte in a way similar to how the analyte binds to a probe on the sensor is provided at a location between the sensor and skin. In one embodiment, the binding solute may be present in a wicking textile (not shown) that brings sweat from skin to the sensors. Because the analyte will bind with the binding solute, the sensor probes are prevented from binding with such analytes. For example, the sensor could be an electrochemical aptamer or antibody sensor, and the binding solute could be an aptamer or antibody that is suspended in solution. Those skilled in the art will recognize other techniques that are useful for lowering concentrations of analytes in sweat such that a more pure fluid is provided for the purposes of calibration.

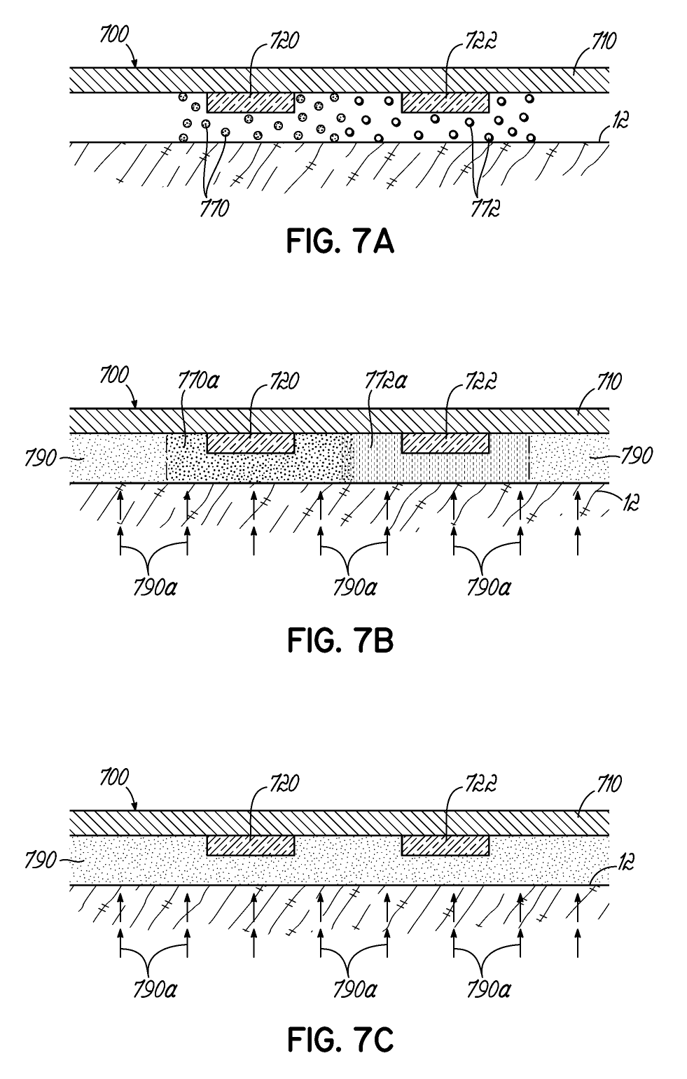

With reference to FIGS. 7A-7C, a device 700 includes a sensor 720 for sensing a first analyte and a sensor 722 for sensing a second analyte, and the device 700 further includes a polymer substrate 710, and calibrants or calibration mediums 770, 772 for calibrating the first and second sensors 720, 722, respectively. The calibration mediums 770, 772 may be positioned adjacent to the sensors 720, 722 using a variety of techniques. For example, the calibration mediums 770, 772 could be a dry powder placed adjacent to a sensor, held in place by a glue or a dissolvable medium, or held in place by another technique until wetted by sweat. The calibration mediums 770, 772 generally: (1) can rapidly take up sweat and allow wetting of sweat against sensors 720, 722; (2) release a concentration of calibrating analytes into sweat near sensors 720, 722 quickly enough to alter the concentration of said analytes in sweat; (3) maintain calibration concentrations of sweat long enough for sensor 720, 722 calibration to be performed; and (4) promote a generally fixed fluid volume initially as they uptake sweat such that calibration analyte concentrations are repeatable. In one embodiment, calibration mediums 770, 722 may be made of a material that would rapidly swell to a known volume as it wets but would more slowly dissolve and wash away, therefore allowing adequate time for calibration (discussed further below). With reference to FIG. 7B, once calibration mediums 770, 772 are wetted with sweat 790 generated as shown by arrows 790a, calibration solutions 770a, 772a are formed. Over time, the calibration analytes within calibration solutions 770a, 772a are transported away from sensors 720, 722 by the sweat 790 such that sensing can be performed on new sweat, as shown in FIG. 7C.

Calibrants, or calibration mediums, useful in embodiments of the present invention can be constructed using a variety of methods. With further reference to FIGS. 7A-7C, calibrants 770, 772 may release the analytes contained therein initially upon contact with sweat or at some time after through time-release techniques. In various embodiments, a calibration medium could be formed from a dissolvable polymer, such as a water soluble polymer or a hydrogel. Exemplary polymers include polyvinylpyrolidone (PVP), polyvinylachohol (PVA), and poly-ethylene oxide. PVP can be used as a dissolvable polymer that can swell with up to 40% water in a humid environment or can be used as a hydrogel if cross-linked using, for example, UV light exposure. Like PVP, PVA can be used as a water dissolvable material or as a hydrogel. Also, such polymers can have a wide range of molecular weights that can affect the rate at which such polymers dissolve. Consider several exemplary embodiments. In one embodiment, a calibration medium of PVP with a known concentration of at least one analyte is coated onto a sensor or is positioned adjacent to a sensor. When wetted or hydrated, the PVP will act as a calibration solution. Such a calibration medium could also contain one or more preservatives. If PVP were or another suitable material were implemented as water dissolvable polymer, and its surface would wet quickly with sweat before it appreciably dissolves. Then, before the PVP fully dissolves, the sweat would hydrate the polymer and allow for sensor calibration. Therefore, the polymer itself could provide a predictable volume and dilution of calibrating analytes confined inside the polymer for a period of time (seconds, or minutes) before it fully dissolves. In one embodiment where the device includes a protein-based sensor, such as an electrically active beacon aptamer sensor, the calibrating analyte confined in the polymer could be a protein, such as a cytokine. Initially, as water and ions from sweat permeate the polymer to wet it, the calibrating concentration of the calibrating protein would remain at least partially immobilized inside the polymer and outside proteins in sweat would be at least partially excluded. Therefore, a predictable dilution or concentration of the calibrant could be provided for a limited period of time long enough for sensor calibration (e.g., on the order of seconds or minutes) before the polymer dissolves. Similarly, hydrogels could be used as calibration mediums as long as a suitable time period for calibration is provided. For example, in one embodiment, the thickness of the hydrogel provides adequate time for the calibrating analyte inside the hydrogel to calibrate the sensor before external analytes in sweat enters the hydrogel and starts to dominate the signal provided from the sensor. It should be recognized that calibration mediums may have alternative configurations. For example, in one embodiment, a calibrant may be constructed of a textile that is coated with analytes. Additionally, various techniques, such as altering the pH, may be used to remove the calibrating analytes from sensors such that they do not interfere with sensing of fresh sweat.

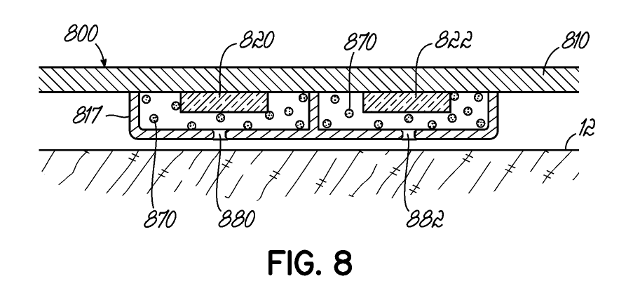

With reference to FIG. 8, a device 800 contains two sensors 820, 822 for example, and two identical calibration mediums 870. Sensors 820, 822 and calibration mediums 870 are enclosed by substrate 810 and seal 817. Seal 817 includes fluidic gates 880, 882. Fluidic gates 880, 882 only allow sweat to reach sensors 820, 822 as determined by the design of the fluidic gates 880, 882 (e.g., based on a dissolution rate of the gate). In one embodiment, when gates 880, 882 allow the passage of fluid, sweat would first enter the space between the membrane 810 and seal 817 and dissolve calibration mediums 870. In this manner, sensors 820, 822 may be calibrated similar to the calibration methods discussed above. After a period of time (e.g., 30 minutes later), the calibration medium 870 would diffuse out through the microfluidic gates 880, 882 as new sweat enters. In this manner, the concentrations of an analyte near the sensors 820, 822 would be dominated by sweat. The device 800 of FIG. 8 is useful when a sensor is to be calibrated and used only when needed. In one embodiment, sensors 820, 822 are one-time use, and the device 800 is configured to perform multiple readings. Where more than one microfluidic gate is used, the gates may be designed to open and close at the same time or at different times. Multiple fluidic gate configurations are possible as known by those skilled in the art, including thermocapillary, electrowetting, melting of wax barriers, or other known techniques. In one embodiment, a wicking element could also be included (not shown) to continue to bring a flow of sweat to the sensor 820 or 822, and mitigate the need for calibration medium to diffuse out and increase the speed at which a reading of analytes in sweat could be made.

With further reference to FIG. 8, in one embodiment, one or both of gates 880, 882 could be a dissolvable polymer (e.g., PVP or PVA) and seal 817 could be a membrane (e.g., a dialysis membrane) that is permeable to water but highly impermeable to at least one analyte to be calibrated. Therefore, as sweat wets the membrane 817, water moves though the membrane 817 and dissolves calibration medium 870 and creates a calibrating solution for calibrating at least one of the sensors 820, 822. Later, as at least one of the gates 880, 882 dissolves away, sweat including the analytes that were previously excluded by membrane 817 enters through the dissolved gate 880, 882 and begins to be sensed by the now-calibrated sensor 820 or sensor 822. It should be recognized that the exact dimensions shown in FIG. 8 are non-limiting and are provided as an example only. For example, in one embodiment, gates 880, 882 could have larger area than membrane 817.

For purpose of clarity, layers and materials in the above-described embodiments of the present invention are illustrated and described as being positioned ‘between’ sweat and sensors and, in some cases, ‘between’ one or more of each layer or material. However, terms such as ‘between’ should not be so narrowly interpreted. The term ‘between’ may also be interpreted to mean ‘in the fluidic pathway of interest’. For example, in one embodiment, a microfluidic channel that is 3 mm long and 300 μm×100 μm in area could be positioned in the pathway (or ‘between’) of flow of sweat from the skin to the sensors and may include any one or more of the features illustrated and discussed for the present invention. Therefore, ‘between’ or other terms should be interpreted within the spirit of the present invention, and alternate embodiments, although not specifically illustrated or described, are included with the present invention so long as they would obviously capture similar purpose or function of the illustrated embodiments.

Electrochemical aptamer-based (“EAB”) biosensor technology, such as is disclosed in U.S. Pat. Nos. 7,803,542 and 8,003,374, may benefit from calibration strategies tailored to their particular shortcomings, biofluid challenges, or other factors. EAB sensors present a stable, reliable bioelectric sensor that is sensitive to target analytes in sweat, while (in the case of MCAS-type sensors) also being capable of multiple analyte capture events during the sensor lifespan. As disclosed in PCT/US17/23399, incorporated by reference in its entirety herein, EAB sensors for use in continuous sweat sensing are configured to provide stable sensor responses with a life cycle extensive enough for multiple analyte binding and release cycles. Such sensors include a plurality of individual aptamer sensing elements, which can repeatedly detect the presence of a molecular target by capturing and releasing target analytes as they establish equilibrium with the aptamer. The sensing element includes an analyte capture complex that includes a selected aptamer, and may include a linking section. The analyte capture complex has a first end covalently bonded to a binding component. This binding component can include a sulfur molecule (thiol), which is in turn covalently bonded to a gold, or other suitable conductive material, electrode base. The analyte capture complex may be bound to the electrode by means of an ethylenediaminetetraacetic acid (EDTA) strain, to improve adhesion in difficult sensing environments, such as sweat biofluid. The sensing element further includes a redox moiety bound to a second end of the analyte capture complex. In the absence of the target analyte, the aptamer is in a first configuration, and the redox moiety is in a first position relative to the electrode. When the sweat sensing device interrogates the sensing element using square wave voltammetry (SWV), the sensing element produces a first electrical signal.

The aptamer in such a sensing element is selected to specifically interact with a target analyte. When the aptamer captures a target analyte molecule, the aptamer undergoes a conformation change that partially disrupts the first configuration and forms a second configuration. The capture of the target analyte accordingly moves the redox moiety into a second position relative to the electrode. Now when the sweat sensing device interrogates the sensing element, the sensing element produces a second electrical signal that is distinguishable from the first electrical signal. After an interval of nanoseconds, milliseconds, seconds or longer, (the “recovery interval”), the aptamer releases the target analyte, and returns to the first configuration, which will produce the corresponding first electrical signal when the sensing element is interrogated.

Current state of the art EAB sensors use a methylene blue (MB) molecule as a redox moiety, because its behavior is well understood, it has a suitably low redox reaction potential, and it is stable during typical electrochemical processes. In testing media with very stable and narrow pH ranges, such as blood, aptamer sensing elements using MB as the redox moiety have very consistent performance through multiple signal-on/signal-off analyte capture cycles. One challenge with the use of EAB sensor technology for sweat sensing, however, is that electrical outputs from such sensors often have a strong dependence on pH. Sweat pH is not stable, and can vary as much as 300 times, from about 4.5 to about 7. Because of the nature of its redox reaction, MB's performance is very sensitive to the variation in biofluid sample pH. Methylene blue's redox potential depends both on its protonation state and its reliance upon a proton (H⁺) transfer to perform the redox reaction. Therefore, MB is doubly sensitive to a biofluid sample's H⁺ concentration, and the pH of a sample must be known to properly correlate a measured signal to a concentration of the target analyte.

In addition to pH dependence, EAB sensor raw signal outputs may vary widely based on individual manufacturing variances between sensors. See PCT/US17/53228, and PCT/US18/35648, which are incorporated by reference herein in their entirety. These variances are due to a number of factors, chiefly differences in the number and/or packing density of aptamer sensing elements that are adhered to the electrode surface of each sensor. Calibration steps may be added to the manufacturing process to improve sensor characterization of individual sensors, individual devices, batches of devices, or manufacturing runs. However, such variation between sensors may also increase over time due to sensor decay where aptamer sensing elements or redox moieties detach from the electrode, and calibration at the time of manufacturing may prove inadequate to address decay caused variations. Another source of sensor-to-sensor variation is non-specific binding with aptamers, which adds a random component to signal output that is unrelated to target analyte concentration. Because of these post-manufacturing sensor-to-sensor variations, devices and methods for periodic or continuous in-use calibration may be required to improve measurement accuracy by EAB sensors.

Due to their sensitivity to pH changes, calibration of EAB sensors while in use is ideally accomplished after the biofluid sample has been pH buffered. Unless otherwise indicated, embodiments of the disclosed invention reflect this preference, even where no buffering apparatus is depicted. Another general preference for EAB sensor calibration is for the calibration media, such as a calibration fluid or gel, to closely match the properties of the biofluid, unless required otherwise. For example, a calibration fluid for EAB sensing in sweat biofluid should match natural sweat's pH, salinity, temperature, and baseline solute content, with the exception of having a specified concentration of the target analyte, e.g., zero target analyte, or maximum physiological target analyte concentration, etc.

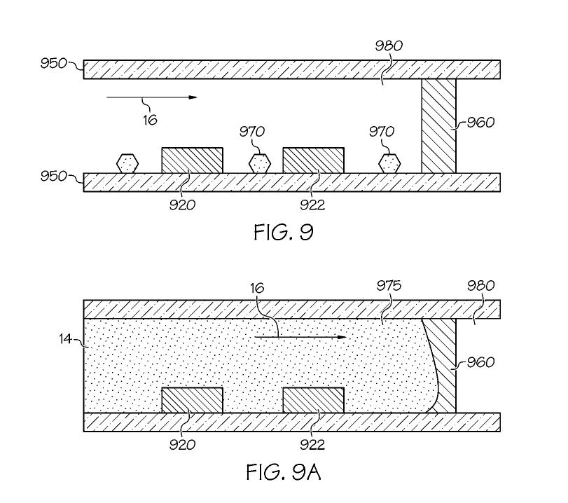

With reference to FIG. 9, at least a portion of a biofluid sensing device configured to perform EAB sensor calibration is depicted. The device includes a fluid impermeable substrate 950, a microfluidic channel 980 (here shown dry, i.e., without a biofluid sample), and one or more electrochemical aptamer-based sensors 920, 922 (two are depicted). The microfluidic channel is in fluidic communication with the skin or other biofluid source, and is configured to move the biofluid from its source, across and away from the sensors 920, 922 in the direction of the arrow 16. The channel 980 is in fluidic communication with an optional source of pH buffering (not shown) located upstream of the sensors relative to the direction of fluid flow 16. This embodiment also includes a dissolvable barrier 960. The dissolvable barrier is comprised of sucrose, or other suitable material that is dissolvable in the biofluid, i.e., is water soluble. Upstream of the barrier, proximate to the sensors 920, 922, the channel contains a dry calibrant 970. The calibrant 970 includes an amount of a target analyte to be measured by the sensors. Given the known volume of the microfluidic channel 980, when dissolved into or mixed with biofluid in the channel, the amount of calibrant provides one of several possible target analyte concentrations, e.g., a zero concentration, a sensor maximum concentration, or a physiological maximum concentration. Ideally, the calibrant 970 is readily dissolvable in the biofluid.

With reference to FIG. 9A, when the device is exposed to a biofluid sample 14, the calibrant dissolves into the biofluid, creating a calibration solution 975 in the microfluidic channel in proximity to the sensors 920, 922. The sensors will then measure the target analyte concentration in the calibration solution. For example, the calibration solution is a sweat sample that has a 100 pM concentration of vasopressin. After an interval, e.g., less than 30 minutes, less than 20 minutes, less than 10 minutes, less than 5 minutes, or less than 1 minute, the barrier 960 will dissolve. This opens a fluid pathway, and the calibration solution 975 will move away from the sensors in the direction of the arrow 16, allowing a biofluid sample to replace the calibration solution, and allowing the sensors to measure the biofluid sample's natural target analyte concentration.

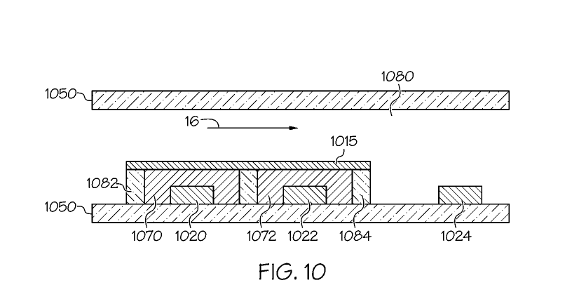

With reference to FIG. 10, an alternate embodiment of at least a portion of a device of the disclosed invention is depicted. In this embodiment, the EAB sensors 1020, 1022, 1024, are configured within a microfluidic channel 1080. One or more first calibration sensors 1020 is located within a first calibration well 1082, which includes biofluid impermeable walls, and a selectively permeable membrane 1015 that is in fluidic communication with the microfluidic channel 1080. The selectively permeable membrane is a least permeable to biofluid and impermeable to a target analyte. The membrane 1015 may also be a dialysis membrane or osmosis membrane, and in some embodiments may facilitate flow of H⁺ and other ions, e.g., Na⁺, Cl⁻, Ca²⁺, HCO₃⁻, Mg²⁺, etc., to regulate pH and salinity. For example, a membrane permeable to biofluid, as well as the biofluid's pH and salinity components, but impermeable to a target analyte, allows the calibration sensors to register the effects of pH and salinity fluctuations on the sensors while a concentration of the target analyte remains constant. In some embodiments, the selectively permeable membrane is one directional, i.e., it allows flow of biofluid into the calibration well(s), and prevents flow of fluid or solutes out of the calibration well(s). The membrane for the first calibration well may be the same as for the second calibration well, or each well may have a separate membrane configured for its purpose. Optionally, one or more second calibration sensors 1022 may be located within a second calibration well 1084. One or more primary sensors 1024 is located in the microfluidic channel to measure the target analyte in the biofluid. While shown downstream of the calibration well(s), relative to the flow of biofluid through the channel, as indicated by the arrow 16, the primary sensor(s) also may be upstream of the calibration well(s), or between calibration wells. The walls of the calibration wells may be made be of the same fluid impermeable material as the substrate 1050, or may be of a different suitable material. Further, the calibration wells may be indentations within the substrate, or constructed on a surface of the substrate so that they protrude fully or partially into the channel.

Within the first calibration well 1082 is a first calibration solution 1070 that is substantially similar to the biofluid, but which has a specified concentration of the target analyte. Within the optional second calibration well 1084, is a second calibration solution 1072 that is also substantially similar to the biofluid, but which has a specified concentration of the target analyte. Various combinations of target analyte concentration within the calibration solutions are possible and contemplated within the disclosed invention. For example, the first calibration solution could be 1) a zero concentration of the target analyte; 2) a physiological minimum, or maximum concentration of the target analyte; 3) a sensor minimum detection, or saturation concentration of the target analyte; 4) a specific concentration of the target analyte; 5) another concentration corresponding to a sensor operational value, such as i_{MAX}, i_{MIN}, or i_{NR}. See PCT/US18/35648, incorporated by reference herein. The second calibration well and solution (if present) also could be any one of the above choices, and also may be selected to perform a function that is complementary to the calibration solution in the first calibration well. Similarly, in some embodiments the calibration well(s) may contain a dry calibrant that absorbs into biofluid or other fluid that crosses the membrane 215. In this way, a variety of different sensor calibrations may be accomplished.

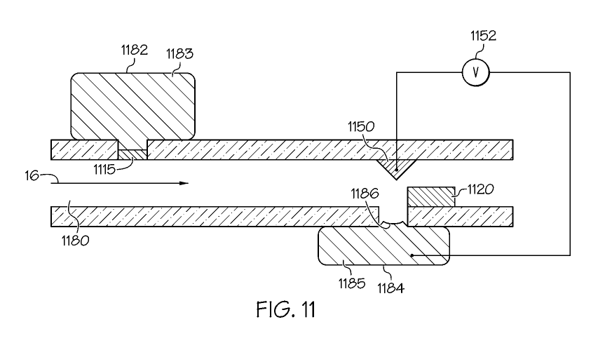

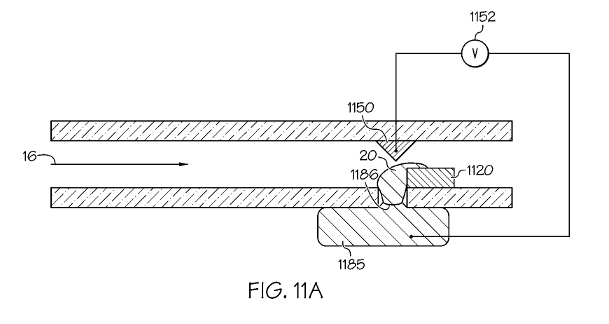

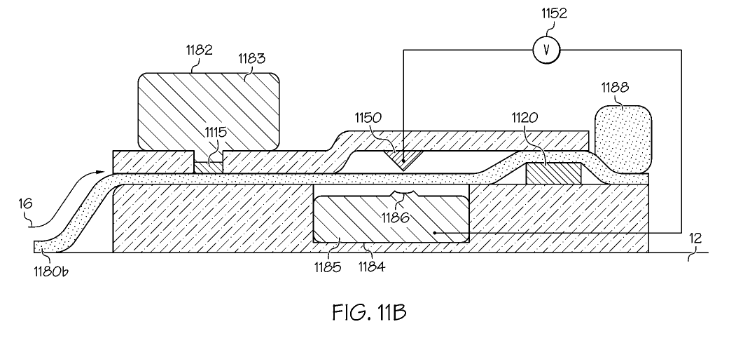

With reference to FIG. 11, at least a portion of an embodiment of the disclosed invention is depicted. This embodiment includes a component for continuous or periodic calibration of one or more EAB sensors. The device includes one or more EAB sensors 1120 (one is depicted) within a microfluidic channel 1180. Some embodiments (see FIG. 11B) may include a microfluidic wick, constructed of a textile or paper, or in some embodiments is a low volume wick 1180b, such as that disclosed in U.S. application Ser. No. 15/958,725, incorporated by reference herein in its entirety, in place of the microfluidic channel. The channel is in fluidic communication with a biofluid source, such as skin, in the case of sweat biofluid. Biofluid moves through the channel in the direction of the arrow 16. Upstream of the sensor 1120 with respect to the fluid flow direction 16 is a pH and/or salinity buffering component, here depicted as a buffering reservoir 1182 that contains a buffering solution 1183. A selectively permeable membrane 1115 allows the exchange of water, ions, and/or other solutes between the buffering solution and biofluid in the channel 1180. Additional devices and methods for buffering a biofluid sample are described in U.S. Provisional Application No. 62/618,778 and PCT/US18/38633, which are incorporated by reference herein in their entirety.

Between the buffering component and the sensor is a calibration component. The calibration component includes a calibration reservoir 1184 that contains a calibration solution 1185. The calibration solution is substantially similar to the biofluid, but contains a specific concentration of a target analyte, e.g., 0 M of the kidney biomarker neutrophil gelatinase-associated lipocalin (NGAL). The calibration reservoir 1184 is in fluidic communication with the channel 1180 via an aperture 1186. The calibration component also includes an electrode 1150 that is in electrical communication with the calibration solution. A current source 1152 creates an electrical potential between the electrode and the calibration solution, so that the electrode has an electrical charge and the calibration solution has an opposite electrical charge. This electrical potential creates an attraction between the calibration solution and the electrode, which draws a droplet of the calibration solution out of the reservoir 1184 and into the channel 1180. The voltages required may be as high as 1 kV, but may be reduced to 200 to 300 V by using techniques known in the art, such as local electrical field shaping. Further voltage reductions to less than 200 V, or less than 100 V are preferred.

With reference to FIG. 11A, once the droplet 20 becomes large enough, it will wet onto the sensor 1120, flooding it with calibration solution 1185, and allowing the sensor to be calibrated. The optional buffering component is not shown.

This calibration arrangement has several advantages. One being the controlled volume of calibration solution delivered to the sensor. The droplet 20 volume is predictable based on the voltage supplied by the current source 1152, as well as the geometry of the aperture 1186. Another benefit of the disclosed embodiment is the ability to periodically re-calibrate the sensor 1120, for example, by periodically applying voltage to the electrode 1150 and solution 1185. Because of their tendency to decay over time, EAB sensors would benefit from such periodic calibration throughout their use.

In addition to calibrating the sensors, the calibration solution can be used to reset an EAB sensor after exposure to target analyte. By flooding the sensor with, for example, a zero concentration of analyte, the calibration component would speed the sensor's release of any captured analyte back into solution, facilitating additional sensing events. Such an arrangement would be particularly useful for resetting EAB sensors configured to measure large molecule concentrations, since larger molecules tend to require longer release times than smaller molecules.

A similar embodiment to the device depicted in FIGS. 11 and 11A is represented by FIG. 11B. This embodiment is shown with a microfluidic wick 1180b instead of a microfluidic channel. The wick is in fluidic communication with a biofluid source (here skin 12 is depicted for the collection of sweat). The embodiment further includes one or more EAB sensors 1120, and an optional microfluidic pump 1188 that may be used to facilitate fluid flow through the device. As the biofluid moves through the device in the direction of the arrow 16, it is in fluidic communication with an optional buffering component via a selectively permeable membrane 1115. The biofluid then moves toward a calibration component, comprising a calibration reservoir 1184, a calibration solution 1185, an aperture 1186, an electrode 1150, and a current source 1152. As in the previous embodiment, the current source 1152 creates a voltage potential between the electrode 1150 and the calibration solution 1185, which draws a droplet (not shown) of calibration solution out of the aperture 1186. Once the droplet becomes large enough, it will contact the wick 1180b, which will then transport a volume of the calibration solution 1185 across the sensor 1120, thereby allowing the sensor to be calibrated. The volume of calibration solution that enters the wick will be determined at least in part by the aperture geometry, and the distance between the aperture and the wick.

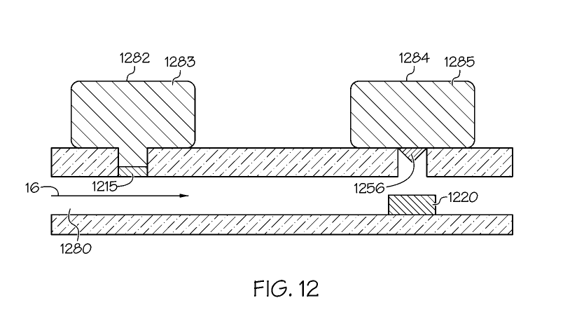

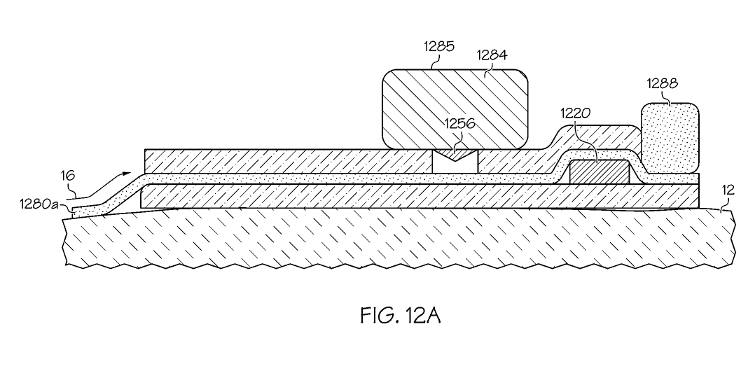

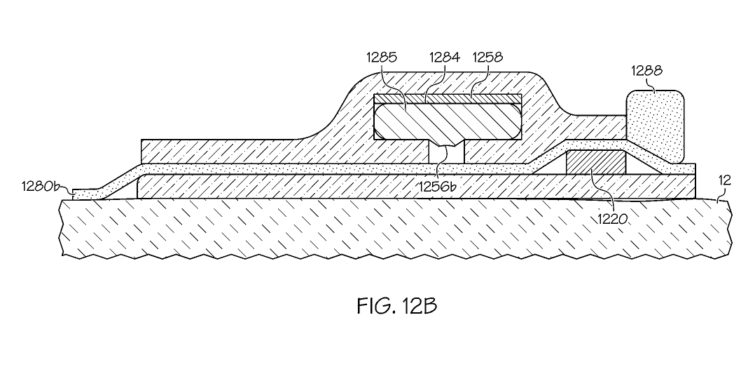

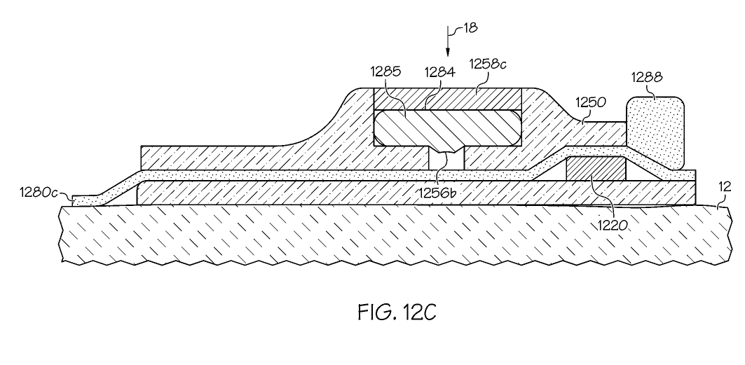

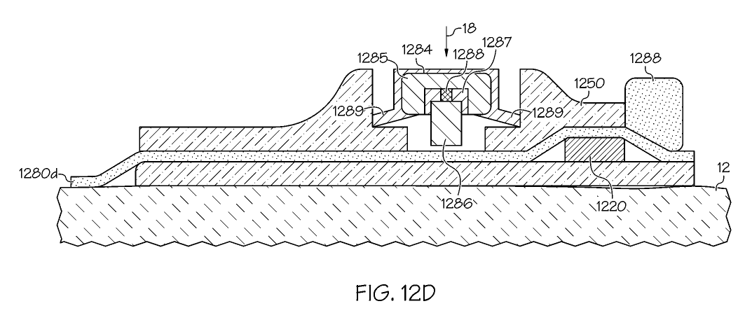

With reference to FIG. 12, a similar embodiment to that depicted in FIGS. 11 to 11B is depicted. In this embodiment, rather than use a current source to manipulate a droplet of calibration solution, this embodiment features a dispenser 1256 configured to deposit a volume of calibration solution into the microfluidic channel 1280. This dispenser 1256 is, e.g., an inkjet nozzle, an electrostatic or electrospray nozzle, a piezo electric nozzle, a thermal nozzle, or other suitable means for spraying or depositing fluid into proximity with the sensor. The dispenser 1256 is separated from biofluid flow in the channel by an air gap, a gate comprising a hydrophobic coating, or an optional membrane (not shown) in order to prevent wicking of calibration solution into the microfluidic channel. With reference to FIG. 12A, an embodiment is depicted featuring a microfluidic wick 1280a in place of the microfluidic channel. In this embodiment, the dispenser is separated from the microfluidic wick by an air gap.

Other arrangements are possible and contemplated under the disclosed invention. For example, see FIG. 12B, instead of using voltages to pull calibration solution into the channel, the calibration reservoir 1284 may have flexible walls, and pressure applied to the reservoir by, e.g., a piezoelectric actuator 1258, or other suitable mechanism, such as a hydraulic or pneumatic actuator. The controlled pressure or displacement distance pushes a volume of calibration fluid 1285 out of an aperture 1256b onto the wick 1280b. With reference to FIG. 12C, a similar embodiment to that depicted in FIG. 12B is shown, wherein the device is actuated manually, rather than by an actuator. Manual actuation is accomplished by directly or indirectly applying pressure to the reservoir 1284, for example, in the direction of the arrow 18 so that a droplet of calibration solution is placed in fluidic communication with the wick 1280c. As pictured, the device substrate 1250 includes an optional flexible section 1258c allowing pressure to be manually communicated to the reservoir. In other embodiments (not shown), manual pressure moves the entire calibration reservoir 1284 closer to the wick 1280c so that a droplet of calibration solution is placed in fluidic communication with the wick 1280c, e.g., the substrate 1250 is flexible and allows the reservoir to move relative to the wick.

With reference to FIG. 12D, another embodiment configured for manual calibration is depicted. In this embodiment, the calibration reservoir 1284 includes a deformable spring component 1289 made of metal, polymer or other suitable material. The spring may have one of a number of configurations, such as a plurality of supporting arms, a flange, one or more coils, etc. The spring 1289 interacts with the substrate 1250 so that in a resting state, an applicator 1286 is separated from the microfluidic wick 1280d by a hydrophobic region, here depicted as an air gap. When pressure is exerted on the reservoir 1284 in the direction of the arrow 18, the spring 1289 deforms, and the applicator 1286 is brought into fluidic contact with the microfluidic wick. The applicator 1286 is configured to hold a set amount of calibration solution, and is made from absorbent material, such as a polymer or paper sponge. The applicator 1286 is in fluidic communication with a microfluidic connector 1288, which is in turn in fluidic communication with the calibration solution 1285. The connector has a high resistance to fluid flow, and substantially prevents fluid flow if the applicator 1286 is saturated with calibration solution. This reduces the influence of actuation duration on the amount of solution that is delivered to the microfluidic wick. When the applicator is less than saturated, the connector 1288 allows a volume of calibration solution to flow into the applicator. The reservoir also includes an attachment component 1287 that holds the applicator and microfluidic connector in place. The attachment component may be a separate component or may be part of the reservoir 1284.

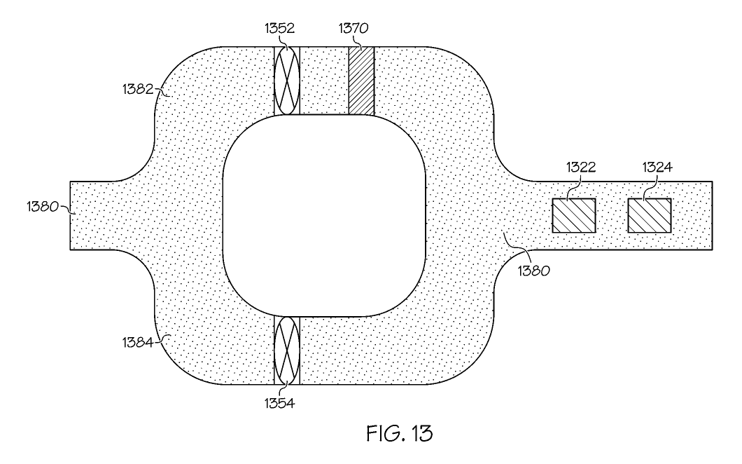

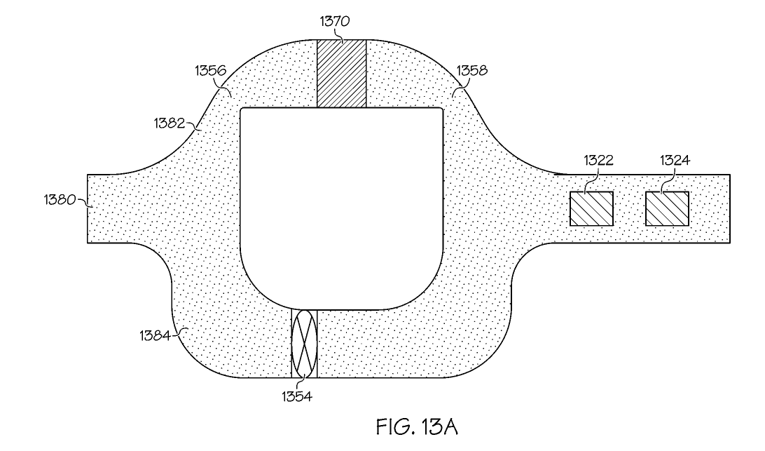

With reference to FIG. 13, in another embodiment, at least a portion of a microfluidic component 1380 is depicted that includes two alternate pathways, a first pathway 1382, and a second pathway 1384, for the transport of biofluid through the device. The microfluidic component may be either a microfluidic channel or a microfluidic wick, as described above. Each pathway has a valve 1352, 1354 to control fluid flow through the pathway. Such valves may be electrical, or pressure actuated valves, may be electrowetting components to allow or stop fluid flow, flaps of wicking paper that are selectively placed in fluidic communication or separated to break fluidic communication, or other suitable arrangement. If the first valve 1352 is opened, and the second valve 1354 is closed, biofluid will flow through the first pathway 1382. Similarly, if the first valve 1352 is closed, while the second valve 1354 is opened, biofluid will be routed through the second pathway 1384. The first pathway is further configured to include a depletion component 1370 that removes a target analyte from the biofluid, while otherwise preserving or leaving unaltered the contents of the biofluid. The depletion component maybe, e.g., a nanofiltration membrane with a cutoff selected to filter out the target analyte. For example, a 100-200 Dalton (Da) filter to remove cortisol from sweat. Other possibilities for depletion components include a gold electrode surface that includes a plurality of aptamer capture elements that remove target analyte from the biofluid, or a quantity of enzyme for breaking down the target analyte. After rejoining the main microfluidic component 1380, the biofluid moves across one or more sensors 1322, 1324. In operation, biofluid is routed through the first pathway 1382 so that the sensors 1322, 1324 are calibrated to a zero concentration of the target analyte. Biofluid is routed through the second pathway 1384 for sensing natural concentrations of target analyte in the biofluid. This configuration may be used to perform periodic calibration or EAB sensor resetting during operation.

With reference to FIG. 13A, an alternate embodiment of the component described in FIG. 13 is depicted that replaces the first valve of that FIG. 1352) with constricted flow areas 1356, 1358. The first pathway 1382 includes a first constricted flow area 1356, and a second constricted flow area 1358, with a depletion component 1370 located within a known volume of channel between constricted flow areas. Closing the valve 1354 forces biofluid to flow through the first pathway 1382, where the depletion component 1370 removes target analyte from the biofluid, and the analyte-depleted biofluid can then move to the one or more sensors 1322, 1324, calibrating them to a zero concentration. Opening the valve 1354 allows biofluid to flow through the second pathway 1384 to the sensor(s) 1322, 1324 for natural target analyte concentration measurement.

In another embodiment (not shown), rather than using a bifurcated channel, a device of the disclosed invention may be configured as a single microfluidic channel or wick capable of a one-time zero-analyte concentration calibration. In such embodiments, a microfluidic channel includes a depletion component located between a biofluid source and an analyte sensor. The depletion component temporarily removes target analyte from the biofluid flowing through the depletion component allowing the device to perform a zero analyte calibration on the sensor. Once the depletion component becomes saturated with target analyte, it ceases to remove additional analyte from the biofluid, allowing the sensor to measure physiological analyte concentrations. Other embodiments may include a depletion component configured as a coating on the sensor itself, or on a biofluid collection component, e.g., a hex wick or concave collector.

This has been a description of the present invention along with a preferred method of practicing the present invention, however the invention itself should only be defined by the appended claims.

What is claimed is:

1. A device, comprising:
one or more electrochemical aptamer-based sensors (each an “EAB sensor”) for measuring a characteristic of an analyte in a biofluid, wherein at least one of the EAB sensors is configured to be calibrated;
a microfluidic component having a volume, and having a first end and a second end, and wherein said microfluidic component is in fluidic communication with a biofluid source, and is selected from one of the following: a microfluidic channel; and a microfluidic wick; and
a calibrant comprising an amount of said analyte.

2. The device of claim 1, further comprising a barrier located between the one or more EAB sensors and the second end of the microfluidic component, wherein said barrier is soluble in the biofluid.

3. The device of claim 1, further comprising:
a calibration well to contain the calibrant, and located between the first end of the microfluidic component and a primary EAB sensor;
a secondary EAB sensor located within the calibration well; and
a selectively permeable membrane in fluidic communication with the microfluidic component and the calibration well.

4. The device of claim 3, further comprising a plurality of calibration wells, wherein each calibration well includes a secondary EAB sensor, and a selectively permeable membrane.

5. The device of claim 1, further comprising a buffering component comprising a buffering solution containing a concentration of at least one ion, and a membrane, wherein said membrane is in fluidic communication with the buffering solution and the microfluidic component, and wherein the buffering component is located between the first end and at least one of the EAB sensors.

6. A device, comprising:
one or more electrochemical aptamer-based sensors (each an “EAB sensor”) for measuring a characteristic of an analyte in a biofluid, wherein at least one of the EAB sensors is configured to be calibrated;
a microfluidic component having a volume, and having a first end and a second end, and wherein said microfluidic component is in fluidic communication with a biofluid source, and is selected from one of the following: a microfluidic channel; and a microfluidic wick;
a calibration solution comprising a known concentration of said analyte, and a solvent, wherein the solvent shares one or more characteristics with the biofluid;
a calibration reservoir for containing the calibration solution; and
a calibration actuator, wherein said actuator facilitates fluidic communication between the calibration solution and a calibrated EAB sensor, and wherein the calibration actuator is located between the first end and the calibrated EAB sensor.

7. The device of claim 6, wherein the calibration actuator is a voltage actuator, comprising:
a voltage source in electrical communication with the calibration solution and an electrode, wherein the voltage actuator creates an electrical potential that draws the calibration solution into fluidic communication with the microfluidic component.

8. The device of claim 6, wherein the calibration actuator is a spray actuator chosen from the following: an inkjet nozzle, an electrostatic nozzle, an electrospray nozzle, a piezo electric nozzle, and a thermal nozzle.

9. The device of claim 6, wherein the calibration actuator is a pressure actuator.

10. A device, comprising:
a microfluidic component for transporting a biofluid, comprising a first end and a second end, and a bifurcation section having a first channel and a second channel, wherein said bifurcation section is located between the first end and the second end;
an analyte depletion component located in the first channel, and configured to remove an amount of an analyte from the biofluid;
a valve located in the second channel;
a flow control component located in the first channel, wherein said flow control component is chosen from one of the following: a valve, and a pair of flow restriction zones, wherein the pair of flow restriction zones comprises a first zone, and a second zone, and wherein the first zone is located between the first end and the analyte depletion component, and the second zone is located between the analyte depletion component and the second end; and
one or more electrochemical aptamer-based sensors (each an “EAB sensor”) for measuring a characteristic of the analyte in the biofluid, and located between the bifurcation section and the second end.
